(19) United States
(12) Patent Application Publication
(10) Pub. No.: US 2015/0150908 A1
(43) Pub. Date: Jun. 4, 2015

(54) COMPOSITIONS AND METHODS FOR TREATING SKIN WOUNDS

(71) Applicant: Team Unlimited, LLC, Boulder, CO (US)

(72) Inventors: Zana Buttermore-Baca, Boulder, CO (US); Chris Leiferman, Boulder, CO (US)

(73) Assignee: Team Unlimited, LLC, Boulder, CO (US)

(21) Appl. No.: 14/559,658

(22) Filed: Dec. 3, 2014

Related U.S. Application Data
(60) Provisional application No. 61/911,037, filed on Dec. 3, 2013.

(51) Int. Cl. A61K 33/40; A61K 33/00; A61K 31/194; A61K 33/14

(52) U.S. Cl. CPC A61K 33/40; A61K 33/14; A61K 33/00; A61K 31/194

(57) ABSTRACT

The invention relates to a composition containing a peroxide, a carbonate source, and a salt. The invention also relates to treating skin wounds using the composition.

### RELATED APPLICATIONS

This application claims priority under 35 U.S.C. §119 from U.S. provisional application No. 61/911,037 entitled “COMPOSITIONS AND METHODS FOR TREATING SKIN WOUNDS,” filed on Dec. 3, 2013, the entire contents of which are fully incorporated by reference herein for all purposes.

### TECHNICAL FIELD

The invention relates to a compositions and methods for treating skin wounds, such as saddle sores.

### BACKGROUND

Bicycling can cause painful saddle sores. Saddle sores are a skin ailment on the buttocks that can be caused by bicycling or horse riding. Bicycle saddles, shorts, and dried salt crystals from sweat act together as an abrasive and cause skin irritation. The irritation can lead to inflamed hair follicles (folliculitis), and if untreated, boils and abscesses. Current popular remedies for saddle sores include NOXZEMA® (which includes camphor, menthol, eucalyptus, and phenol), products containing benzocaine and resorcinol, PREPARATION H® (which includes glycerin, phenylephrine, pramoxine, and white petrolatum), tea tree oil, antibacterial ointments, TEGADERM™, talcum powder, baby powder, products containing talc or cornstarch, and calamine. None of these remedies provides completely satisfactory result. As a result, there is a need in the art for improved treatments for skin wounds, especially saddle sores.

### SUMMARY

Provided herein is a composition comprising a peroxide, a carbonate source, a salt, a thickening agent, and water. The peroxide may be a peroxide ion, an inorganic peroxide, an organic peroxide, or an organic hydroperoxide. The inorganic peroxide may be hydrogen peroxide. The carbonate source may be ammonium bicarbonate, cesium bicarbonate, calcium bicarbonate, magnesium bicarbonate, potassium bicarbonate, or sodium bicarbonate. The salt may be magnesium sulfate, sodium chloride, borax, sodium citrate, or ammonium chloride. The thickening agent may be a carbomer, which may be a carbomer 940. The pH of the composition may be about 5.0-6.0, and may be about 5.6. The composition may further comprise an acid, which may be used to adjust the pH. The acid may be a weak organic acid, and may be lactic acid, acetic acid, formic acid, citric acid, oxalic acid, or uric acid.

Further provided herein is a composition consisting essentially of hydrogen peroxide, sodium chloride, sodium bicarbonate, citric acid, carbomer 940, and water. The pH of the composition may be 5.6. The composition may consist essentially of 9.1% (v/v) sodium carbonate, 9.1% (v/v) sodium chloride, 9.1% (v/v) hydrogen peroxide, wherein the hydrogen peroxide is a 3% solution of hydrogen peroxide, 9.1% (v/v) citric acid, 9.1% (v/v) carbomer 940, and 54.5% (v/v) water.

Also provided herein is a method for treating skin wound in a patent, which may comprise topically administering the composition to a patient in need thereof. The skin wound may be a saddle sore. The skin wound may also be a boil, an abscess, a sore, acne vulgaris, a skin rash, or a skin infection. The skin rash may be caused by sweat. The skin infection may be caused by chafing or pressure, or a bacterial infection.

### DETAILED DESCRIPTION

The inventors have found that a composition comprising a peroxide, a carbonate source, and a salt is surprisingly effective at treating skin wounds. The composition may comprise water. In addition, the pH of the composition may be adjusted with an acid, so that the composition further comprises an acid. In particular, an aqueous solution of hydrogen peroxide, sodium bicarbonate, and sodium chloride, at a pH of about 5.6 using citric acid to adjust the pH, is especially effective. The inventors have also discovered that including a thickening agent, such as carbomer 940, provides superior properties for topical application.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

“About” means an acceptable error for a particular value as determined by one of ordinary skill in the art, which depends in part on how the value is measured or determined. “About” may mean within 1, 2, 3, or 4 standard deviations. “About” may also mean within 50%, 20%, 15%, 10%, 9%, 8%, 7%, 6%, 5%, 4%, 3%, 2%, 1%, 0.5%, or 0.05% of a given value or range.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

“Treatment” or “treating,” when referring to protection of an animal from a disease or condition, means preventing, suppressing, repressing, or completely eliminating the disease or condition. Preventing the disease or condition involves administering a composition of the present invention to an animal prior to onset of the disease or condition. Suppressing the disease or condition involves administering a composition of the present invention to an animal after induction of the disease or condition but before its clinical appearance. Repressing the disease or condition involves administering a composition of the present invention to an animal after clinical appearance of the disease or condition.

### 2. Sore Treatment Composition

Provided herein is a composition, which may comprise a peroxide, a carbonate source, and a salt. The composition may comprise water. The composition may also comprise a thickening agent. The composition may further comprise an acid, which may adjust the pH. The composition may be an aqueous solution, a salve, a balm, a lotion, a cream, an ointment, a paste, or a gel. The composition may consist essentially of water, the peroxide, the carbonate source, the salt, the acid, and the thickening agent. The water may be filtered or distilled. The composition may exclude pharmaceutically active ingredients, including antibiotics, antiseptic agents, analgesics, anti-inflammatory agents, anti-infective agents, and proteolytic enzyme wound debriders.

a. Peroxide

The peroxide may be a compound comprising an oxygen-oxygen single bond, or a peroxide anion (O₂²). The peroxide may be a peroxide ion, an inorganic peroxide, an organic peroxide (R₁—O—O—R₂, where R₁ and R₂ are each independently a hydrocarbon moiety), an organic hydroperoxide (R—O—O—H, where R is a hydrocarbon moiety), or a peracid (R—C(═O)——O—O—H, where R is a hydrocarbon moiety). The inorganic peroxide may be an ionic or a covalent peroxide. The ionic peroxide may be a peroxide of an alkali or alkaline earth metal. The covalent peroxide may be hydrogen peroxide or peroxymonosulfuric acid. The peroxide may also be a peroxide of a transition metal. In particular, the peroxide may be hydrogen peroxide. The amount of peroxide in the composition may be equivalent to about 4.0, 4.5, 5.0, 5.5, 6.0, 6.5, 7.0, 7.5, 8.0, 8.5, 8.6, 8.7, 8.8, 8.9, 9.0, 9.1, 9.2, 9.3, 9.4, 9.5, 9.6, 10.0, 10.5, 11.0, 11.5, 12.0, 12.5, 13.0, 13.5, 14.0, 14.5, or 15.0% (v/v) of a 3% peroxide solution. The amount may also be equivalent to between about 8.5-9.5% or 8.0-10.0% (v/v) of a 3% peroxide solution. In particular, the amount may be equivalent to about 9.1% (v/v) of a 3% peroxide solution.

b. Carbonate Source

The carbonate source may be a bicarbonate, which may be ammonium bicarbonate, cesium bicarbonate, calcium bicarbonate, magnesium bicarbonate, potassium bicarbonate, sodium sesquicarbonate, or sodium bicarbonate. In particular, the carbonate may be sodium bicarbonate. The amount of the carbonate source in the composition may be equivalent to about 4.0, 4.5, 5.0, 5.5, 6.0, 6.5, 7.0, 7.5, 8.0, 8.5, 8.6, 8.7, 8.8, 8.9, 9.0, 9.1, 9.2, 9.3, 9.4, 9.5, 9.6, 10.0, 10.5, 11.0, 11.5, 12.0, 12.5, 13.0, 13.5, 14.0, 14.5, or 15.0% (v/v) of a substantially pure solid of the carbonate source. The amount may also be equivalent to between about 8.5-9.5% or 8.0-10.0% (v/v) of a substantially pure solid of the carbonate source. In particular, the amount may be equivalent to about 9.1% (v/v) of a substantially pure solid of the carbonate source.

c. Salt

The salt may be an inorganic salt. The inorganic salt may be magnesium sulfate, sodium chloride, borax, sodium citrate, or ammonium chloride. In particular, the salt may be sodium chloride. The amount of the salt in the composition may be equivalent to about 4.0, 4.5, 5.0, 5.5, 6.0, 6.5, 7.0, 7.5, 8.0, 8.5, 8.6, 8.7, 8.8, 8.9, 9.0, 9.1, 9.2, 9.3, 9.4, 9.5, 9.6, 10.0, 10.5, 11.0, 11.5, 12.0, 12.5, 13.0, 13.5, 14.0, 14.5, or 15.0% (v/v) of a substantially pure solid of the salt. The amount may also be equivalent to between about 8.5-9.5% or 8.0-10.0% (v/v) of a substantially pure solid of the salt. In particular, the amount may be equivalent to about 9.1% (v/v) of a substantially pure solid of the salt.

d. Thickening Agent

The thickening agent may be acacia, alginic acid, bentonite, a carbomer, carboxymethylcellulose, ethylcellulose, gelatin, hydroxyethylcellulose, hydroxypropyl cellulose, magnesium aluminum silicate (VEEGUM®), methylcellulose, poloxamers (PLURONICS®), polyvinyl alcohol, sodium alginate, tragacanth, or a xanthan gum. The carbomer may be a carbomer 934, 940,941, 980, 981, 1342, 1382, 2984, or 5984. The carbomer 940 may have a Brookfield viscosity of 40,000-60,000 mPa•s at a concentration of 0.5% in a neutralized solution at 25° C. The carbomer 940 may be CARBOPOL® 940. The carbomer may also be CARBOPOL® Ultrez 10, which may have a Brookfield viscosity of 45,000-65,000 mPa•s at a concentration of 0.5%, pH 7.3-7.8, at 25° C. In particular, the thickening agent may be a carbomer 940. The amount of the thickening agent in the composition may be equivalent to about 4.0, 4.5, 5.0, 5.5, 6.0, 6.5, 7.0, 7.5, 8.0, 8.5, 8.6, 8.7, 8.8, 8.9, 9.0, 9.1, 9.2, 9.3, 9.4, 9.5, 9.6, 10.0, 10.5, 11.0, 11.5, 12.0, 12.5, 13.0, 13.5, 14.0, 14.5, or 15.0% (v/v) of a substantially pure solid of the thickening agent. The amount may also be equivalent to between about 8.5-9.5% or 8.0-10.0% (v/v) of a substantially pure solid of the thickening agent. In particular, the amount may be equivalent to about 9.1% (v/v) of a substantially pure solid of the thickening agent.

e. pH
(1) pH Level

The pH of the composition may be suitable for application of the composition to the skin of a mammal. The pH may be about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, or 6.5. The pH may also be about 5.0-6.0. In particular, the pH may be about 5.6.

### (2) Acid for pH Adjustment

The acid may be an organic acid, which may be a weak organic acid. The acid may be lactic acid, acetic acid, formic acid, citric acid, oxalic acid, or uric acid. The amount of the acid in the composition may be equivalent to about 4.0, 4.5, 5.0, 5.5, 6.0, 6.5, 7.0, 7.5, 8.0, 8.5, 8.6, 8.7, 8.8, 8.9, 9.0, 9.1, 9.2, 9.3, 9.4, 9.5, 9.6, 10.0, 10.5, 11.0, 11.5, 12.0, 12.5, or 13.0% (v/v) of a substantially pure solid of the acid. The acid concentration may also be an amount equivalent to between about 8.5-9.5% or 8.0-10.0% (v/v) of a substantially pure solid of the acid. In particular, the acid concentration may be an amount equivalent to about 9.1% (v/v) of a substantially pure solid of the acid.

f. Formulation

The composition may be impregnated into a dressing, which may be a bandage, wound protective dressing, foam, sponge, pad, gauze, collagen, film dressing, patch, drape, or paste.

### 3. Methods of Making Sore Treatment

The composition may be made by mixing water, the peroxide, the carbonate source, the acid, and the salt, at ambient temperature (i.e., 25° C.). Each ingredient may mixed into the composition as an aqueous solution or as a solid. The ingredients may be mixed by any methods known in the art. The peroxide and the carbonate source may be mixed together in a first mixture, and the thickening agent and water may be mixed together in a second mixture. The first and second mixtures may be mixed to form a third mixture. The salt may be added to the third mixture to form a fourth mixture. The pH of the fourth mixture may be adjusted, which may be accomplished by adding the acid.

### 4. Method of Treating a Skin Wound

Provided herein is a method of treating a skin wound in a patient in need thereof. The skin wound may be a cut, an abrasion, a laceration, a burn, or a sore. The sore may be caused by friction or chafing of the skin. The sore may be a blister or a saddle sore, which may be caused by friction resulting from bicycling or horse riding. The skin wound may also be a boil or abscess, acne vulgaris (such as pimples), skin rash, or an infection. The skin rash may be caused by sweat. The infection may be caused by chafing or pressure, such as from sweaty clothes, or bacteria. The patient may be a mammal, which may be a dog, cat, horse, cow, pig, or human.

The treatment may comprise administering the composition to the skin of the patient. The composition may be administered topically once, twice, or 3, 4, 5, 6, 7, 8, 9, 10, 11, or 12 times per day. The composition may also be administered 1-2, 1-4, 1-6, 1-8, 2-4, 2-6, 2-8, 4-6, or 4-8 times per day. The composition may be topically administered for about 15, 30, 45, or 60 min., or 1, 2, 3, 4, 5, 6, 7, 8, 9, 10, 11, 12, 16, 18, 24, or 48 h. The composition may also be administered for about 15 min. to 24 h, 15 min. to 1 h, 30 min. to 1 h, 1 to 24 h, 1 to 48 h, 1 to 4 h, 1 to 6 h, 2 to 4 h, 2 to 8 h, 4 to 8 h, 4 to 12 h, 8 to 12 h, 8 to 24 h, 12 to 24 h, or 24 to 48 h.

Repeated administration of the composition may be continued for 1, 2, 3, 4, 5, 6, 7, 8, 9, 10, 11, 12, 13, or 14 days. The repeated administration may also be continued for 1-3, 1-7, 4-7, 4-10, 7-10, 7-14, or 10-14 days. After application of the composition, the skin wound may be covered with a dressing, which may be a bandage, wound protective dressing, foam, sponge, pad, gauze, collagen, film dressing, patch, or drape.

The present invention has multiple aspects, illustrated by the following non-limiting examples.

### EXAMPLE 1

### Making a Saddle Sore Treatment

This examples shows how one variant of the claimed composition was made. The composition contained the following ingredients.

| | Original | Volume Added | Final Amount |
|---|---|---|---|
| Ingredient | concentration | (mL) | % (v/v) |
| Hydrogen peroxide | 3% aqueous | 14.79 | 9.1 |
| | solution | | |
| Sodium chloride | n/a (substantially | 14.79 | 9.1 |
| | pure solid) | | |
| Sodium bicarbonate | n/a (substantially | 14.79 | 9.1 |
| | pure solid) | | |
| Citric acid | n/a (substantially | 14.79 | 9.1 |
| | pure solid) | | |
| Carbomer 940 | n/a (substantially | 14.79 | 9.1 |
| | pure solid) | | |
| Water | n/a | 88.74 | 54.5 |

Equal volumes (14.79 mL each) of an aqueous solution of 3% hydrogen peroxide (TARGET®) and finely ground solid sodium bicarbonate (i.e., baking soda) (ARM AND HAMMER®) were mixed together. Separately, 88.74 mL of filtered water and 14.79 mL of carbomer 940 (purchased in bulk from Ingredients to Die For) were mixed together and the carbomer was allowed to completely saturate to form a gel. Next, the hydrogen peroxide and sodium bicarbonate solution was thoroughly mixed with the carbomer gel, and 14.79 mL finely ground solid sodium chloride (sea salt purchased in bulk from Sprouts) was then added. This mixture was then mixed constantly in order to subside a slight foam created from the resulting reaction. To adjust the pH, 14.79 mL solid citric acid (purchased in bulk from Spicy World) was added into the mixture, and the solution was then repeatedly stirred until the ingredients were fully dissolved.

1. A composition comprising a peroxide, a carbonate source, a salt, a thickening agent, and water.

2. The composition of claim 1, wherein the peroxide is selected from the group consisting of a peroxide ion, an inorganic peroxide, an organic peroxide, and an organic hydroperoxide.

3. The composition of claim 2, wherein the inorganic peroxide is hydrogen peroxide.

4. The composition of claim 1, wherein the carbonate source is selected from the group consisting of ammonium bicarbonate, cesium bicarbonate, calcium bicarbonate, magnesium bicarbonate, potassium bicarbonate, and sodium bicarbonate.

5. The composition of claim 4, wherein the carbonate source is sodium bicarbonate.

6. The composition of claim 1, wherein the salt is selected from the group consisting of magnesium sulfate, sodium chloride, borax, sodium citrate, and ammonium chloride.

7. The composition of claim 1, wherein the thickening agent is a carbomer.

8. The composition of claim 7, wherein the carbomer is carbomer 940.

9. The composition of claim 1, wherein the pH is about 5.0-6.0.

10. The composition of claim 9, wherein the pH is about 5.6.

11. The composition of claim 9, wherein the composition further comprises an acid to adjust the pH.

12. The composition of claim 11, wherein the acid is a weak organic acid.

13. The composition of claim 12, wherein the weak organic acid is selected from the group consisting of lactic acid, acetic acid, formic acid, citric acid, oxalic acid, and uric acid.

14. The composition of claim 13, wherein the weak organic acid is citric acid.

15. A composition consisting essentially of hydrogen peroxide, sodium chloride, sodium bicarbonate, citric acid, carbomer 940, and water.

16. The composition of claim 15, wherein the pH of the composition is about 5.6.

17. The composition of claim 15, wherein the composition consists essentially of 9.1% (v/v) sodium carbonate, 9.1% (v/v) sodium chloride, 9.1% (v/v) hydrogen peroxide, wherein the hydrogen peroxide is a 3% solution of hydrogen peroxide, 9.1% (v/v) citric acid, 9.1% (v/v) carbomer 940, and 54.5% (v/v) water.

18. A method for treating a skin wound in a patient, comprising topically administering the composition of claim 1 to a patient in need thereof.

19. The method of claim 18, wherein the skin wound is a saddle sore.

20. The method of claim 18, wherein the skin wound is selected from the group consisting of a boil, an abscess, a sore, acne vulgaris, a skin rash, and a skin infection.

21. The method of claim 20, wherein the skin rash is caused by sweat.

22. The method of claim 20, wherein the skin infection is caused by chafing or pressure, or a bacterial infection.
